SEQUENCE LISTING (14 sequences)

SEQ ID NO: 1 - DNA, 21 bp, Artificial Sequence
  C3aR primer sequence Mouse F
cagggaaaagtcagtgctcag

SEQ ID NO: 2 - DNA, 20 bp, Artificial Sequence
  C3aR primer sequence Mouse R
ggccgtgagtgtaggtcagt

SEQ ID NO: 3 - DNA, 20 bp, Artificial Sequence
  GAPDH primer sequence Mouse F
agaacatcatccctgcatcc

SEQ ID NO: 4 - DNA, 21 bp, Artificial Sequence
  GAPDH primer sequence Mouse R
ataccaggaaatgagcttgac

SEQ ID NO: 5 - DNA, 20 bp, Artificial Sequence
  C3aR primer sequence Human F
cccactgtccctcaaacaat

SEQ ID NO: 6 - DNA, 19 bp, Artificial Sequence
  C3aR primer sequence Human R
aagtccgctgctcaccata

SEQ ID NO: 7 - DNA, 18 bp, Artificial Sequence
  GAPDH Primer Sequence Human F
ggaaactgtggcgtgatg

SEQ ID NO: 8 - DNA, 18 bp, Artificial Sequence
  GAPDH Primer Sequence Human R
tgggtgtcgctgttgaag

SEQ ID NO: 9 - DNA, 15 bp, Artificial Sequence
  iNOS primer sequence Mouse F
gagttgtggattgtc

SEQ ID NO: 10 - DNA, 20 bp, Artificial Sequence
  iNOS primer sequence Mouse R
gtgagggcttggctgagtga

SEQ ID NO: 11 - DNA, 20 bp, Artificial Sequence
  IL-6 primer sequence Mouse F
tccggagaggagacttcaca

SEQ ID NO: 12 - DNA, 22 bp, Artificial Sequence
  IL-6 primer sequence Mouse R
tccagtttggtagcatccatca

SEQ ID NO: 13 - DNA, 19 bp, Artificial Sequence
  TNF-A primer sequence Mouse F
ggcatggatctcaaagaca

SEQ ID NO: 14 - DNA, 18 bp, Artificial Sequence
  TNF-A primer sequence Mouse R
tgggctcataccagggtt

(19) United States
(12) Patent Application Publication
(10) Pub. No.: US 2022/0287998 A1
(43) Pub. Date: Sep. 15, 2022

(54) USE OF C3aR IN PREPARING DRUG FOR PREVENTING OR TREATING POLYCYSTIC KIDNEY DISEASE

(71) Applicant: Shanghai Changzheng Hospital, Shanghai (CN)

(72) Inventors: Changlin Mei, Shanghai (CN); Shuwei Song, Shanghai (CN); Can He, Shanghai (CN); Lili Fu, Shanghai (CN); Beilin Su, Shanghai (CN)

(73) Assignee: Shanghai Changzheng Hospital, Shanghai (CN)

(21) Appl. No.: 17/199,999

(22) Filed: Mar. 12, 2021

(51) Int. Cl. A61K 31/198; A61K 31/7105; A61P 13/12; A61K 31/713

(52) U.S. Cl. CPC A61K 31/198; A61K 31/7105; A61P 13/12; A61K 31/713

(57) ABSTRACT

The disclosure provides a use of C3aR in preparing drugs for preventing or treating polycystic kidney disease, belonging to the technical field of medicines. The disclosure discovers that C3aR inhibitor can down-regulate or reduce the C3aR level by inhibiting proliferation of cyst epithelial cells, blocking chemotaxis of C3a-C3aR on macrophages, inhibiting the interaction between C3a and C3aR, and expressing the p-ERK, p-P65 proteins, and then prevent cyst growth, improve the inflammatory state, thus treating polycystic kidney disease. The C3aR inhibitor can be used for the development of novel lead compounds for preventing or treating autosomal dominant polycystic kidney disease, and also can be used for preparing drugs for preventing or treating polycystic kidney disease.

### FIELD OF THE INVENTION

The present disclosure belongs to the technical field of medicines, and particularly relates to a use of C3aR in preparing drug for preventing or treating polycystic kidney disease.

### BACKGROUND OF THE INVENTION

Autosomal dominant polycystic kidney disease (ADPKD) is the most common single-gene inherited nephropathy, its two major pathogenic genes are mainly pKD1 and PKD2. Any gene pathogenic mutation can cause multiple kidney cysts. Kidney cysts grow progressively and destroy the structure and function of kidney. At the age of 60, 50% of the patients enter end-stage kidney failure, the life of which can only be maintained by dialysis or kidney transplantation. Since the pathogenesis of ADPKD is complex and has not been elucidated so far, there is a lack of safe and effective therapeutic medications for treating ADPKD. In recent years, more and more attention has been paid to the role of complement-inflammation in the pathogenesis and progression of ADPKD. Our previous studies show that the expression of factors such as complement B and C9 in kidney cysts of ADPKD patients and Han:sPRD rats is up-regulated, the complement replacement pathway is abnormally activated, and the infiltration of kidney interstitial macrophages is increased; the treatment of Han:sPRD rats and PKD1 knockout mice with the complement inhibitor such as rosmarinic acid or cobalt chloride can significantly reduce the infiltration of kidney interstitial macrophages, inhibit the growth of kidney cysts, and improve kidney function. As an important component of the complement system, complement C3 can be activated for producing C3a, which can promote inflammatory cell infiltration and stimulate inflammatory cells to produce inflammatory factors. However, the expression of C3a-C3a receptor (C3aR) in polycystic kidney and its effect on macrophages have not been reported.

SB290157 is a C3aR non-peptide inhibitor identified from high throughput screening in 2001 by Ames etc., which inhibits intracellular Ca mobilization and chemotaxis induced by C3a by specifically blocking C3a binding to C3aR, without affecting other complement components. Although SB290157 can broadly inhibit C3aR in humans, mice, rats, and the like, the identity (only 60% to 65%) of C3aR in these species is relatively low. In terms of animal experiments, some scholars have used SB290157 to treat diabetic nephropathy, kidney fibrosis, essential hypertension, cerebral ischemia-reperfusion, etc., all of which have achieved good efficacy and no serious adverse reactions have been found. However, SB290157 has not been reported in polycystic kidney disease.

### SUMMARY OF THE INVENTION

In view of this, the purpose of the present disclosure is to provide a use of C3aR and its inhibitor SB290157 in preparing drug for preventing or treating polycystic kidney disease, thus filling the blank in the field of therapeutic drugs for polycystic kidney disease.

In order to achieve the foregoing purpose, the present disclosure provides the following technical solutions:

the present disclosure provides a use of C3aR serving as drug target in preparing drug for preventing and/or treating polycystic kidney disease.

Preferably, the polycystic kidney disease comprises autosomal dominant polycystic kidney disease and autosomal recessive polycystic kidney disease.

Preferably, the drug for preventing and/or treating polycystic kidney disease is capable of inhibiting the activity of C3aR and/or down-regulating expression of C3aR gene.

The present disclosure also provides a use of an agent for inhibiting the activity of C3aR and/or down-regulating the expression of C3aR gene in preparing drug for preventing or treating polycystic kidney disease.

Preferably, the agent for inhibiting the activity of C3aR and/or down-regulating the expression of C3aR gene includes but is not limited to:

proteins specifically binding to C3aR;

siRNA molecules, miRNA molecules or antisense nucleotides capable of specifically interfering expression and processing of C3aR gene;

C3aR inhibitors, antagonists, downregulators, retardants or blockers.

Preferably, the C3aR inhibitors comprise SB290157 and compounds with similar or identical activity functions to SB290157.

Preferably, the C3aR inhibitor acts through any one or several of the following ways:

inhibiting proliferation of cyst epithelial cells; blocking chemotaxis of C3a-C3aR on macrophages; inhibiting the interaction between C3a and C3aR; and lowering the C3aR level.

Preferably, the C3aR inhibitor down-regulates the expression of p-ERK and p-P65 proteins by inhibiting the interaction between C3a and C3aR and inhibiting the activation of ERK and NF-kB pathways.

The present disclosure also provides a use of C3aR as drug target in screening drugs for preventing and/or treating polycystic kidney disease, wherein the drugs for preventing and/or treating polycystic kidney disease can inhibit the activity of C3aR and/or down-regulate the expression of C3aR gene.

The present disclosure also provides a drug for preventing and/or treating polycystic kidney disease, comprising an active ingredient capable of inhibiting the activity of C3aR and/or down-regulating the expression of C3aR gene.

Compared with the prior art, technical solutions in the present disclosure has the following beneficial effects:

According to the present disclosure, it is found for the first time that C3a-C3aR are highly expressed in both the ADPKD patients and the polycystic kidney mouse model (Pkd1 conditional knockout mouse), and the expression of C3a-C3aR is positively correlated with the disease progression to a certain extent, suggesting that C3a-C3aR may be involved in the ADPKD progression. Then, according to the present disclosure, it is found that the C3aR inhibitor SB290157 can significantly improve the kidney function and cyst-related indexes of Pkd1 knockout mouse, resulting in limiting the proliferation of cyst cells, increasing the apoptosis, and down-regulating the expression of multiple ADPKD-related pathway proteins, which indicates that the inhibition of C3a-C3aR may have a therapeutic effect on ADPKD.

In an in vitro experiment of the present disclosure, it is found that there is co-localization of C3aR and macrophage marker F4/80 in the kidney tissue of Pkd1 knockout mouse, with C3aR expressed on the macrophage membrane, and it is further found that the inhibition of C3aR can effectively reduce macrophage infiltration in the mouse polycystic kidney tissue, thereby indicating that the C3a-C3aR may have chemotactic effect on the macrophages of the ADPKD kidney. The present disclosure further finds that C3aR is mainly expressed in M1-type macrophages in subsequent experiments, and C3a-C3aR may promote the production of inflammatory factors (such as TNF-α and the like) by activating Akt, ERK, NF-kB and STAT1 signaling pathways in M1-type macrophages.

In summary, the present disclosure firstly discovers that the increase of C3a in the polycystic kidney tissue causes macrophage infiltration and activation through C3aR so as to promote ADPKD progression, and the mechanism may be caused by Akt activation and increased TNF-α production. C3aR inhibitor is a novel target for the treatment of ADPKD.

### BRIEF DESCRIPTION OF THE DRAWINGS

FIG. 1 is a graph showing C3a level in the kidney tissue and serum of wild-type and Pkd1 conditional knockout mice, wherein A depicts C3a level in the kidney tissue, B depicts C3a level in serum, expressed as mean±SEM, * means P<0.05, ** means P<0.01, ns means P>0.05;

FIG. 2 shows the relative expression of C3aR protein in the kidney tissue of wild-type and Pkd1 conditional knockout mice, wherein A depicts a Western blot image, and B depicts a gray scale analysis image of C3aR expression, expressed as mean±SEM, ** means P<0.01;

FIG. 3 shows the relative expression of C3aR in the kidney tissue of wild-type and Pkd1 conditional knockout mice detected by immunohistochemistry (×400);

FIG. 4 shows the relative expression of C3aR mRNA in the kidney tissue of wild-type and Pkd1 conditional knockout mice detected by RT-PCR, expressed as mean±SEM, ** means P<0.01;

FIG. 5 shows the relative expression of C3aR protein in the kidney tissue of normal human and ADPKD patients, wherein A depicts a Western blot image, B depicts a gray scale analysis image of C3aR expression, expressed as mean±SEM, ** means P<0.01;

FIG. 6 shows the relative expression of C3aR in the kidney tissue of normal human and ADPKD patients detected by immunohistochemistry (×400);

FIG. 7 shows the relative expression of C3aR mRNA in the kidney tissue of normal human and ADPKD patients detected by RT-PCR, expressed as mean±SEM, ** means P<0.01;

FIG. 8 shows the immunofluorescence observation of the localization of C3aR and F4/80 in the kidney tissue of polycystic kidney mice (×400);

FIG. 9 shows immunofluorescence staining for F4/80 in the kidney tissue of each group of mice (×400);

FIG. 10 shows the relative expression of C3aR protein in different types of macrophages, where A depicts a Western blot image; B depicts a gray scale analysis image of protein expression in each group, expressed as mean±SEM, ** means P<0.01 compared with the control group, ## means P<0.01 compared with the LPS group; C depicts the relative expression of C3aR in different types of macrophages detected by immunofluorescence (×400); D depicts the relative expression of C3aR mRNA in different types of macrophages, expressed as mean±SEM, ** means P<0.01 compared with the control group; ## means P<0.01 compared with the LPS group;

FIG. 11 shows a comparison of the relative expression of iNOS, IL-6, TNF-α mRNA in macrophages from different treatment groups, expressed as mean±SEM, ** means P<0.01 compared with the control group, # means P<0.05 compared with the LPS group (##: p<0.01), means P<0.01 compared with the LPS+C3a group;

FIG. 12 shows a comparison of TNF-α levels in macrophage culture supernatants from different treatment groups, expressed as mean±SEM, ** means P<0.01 compared with the control group, # means P<0.05 compared with the LPS group (##: p<0.01); means P<0.01 compared with the LPS+C3a group;

FIG. 13 shows a comparison of the relative expression of p-Akt, p-ERK, p-P65, p-STAT1 and TNF-α proteins in macrophages from different treatment groups, wherein A depicts a Western blot image; b depicts a gray scale analysis image of protein expression in each group, expressed as mean±SEM, ** means P<0.01 compared with the control group, ## means P<0.01 compared with the C3a group, means P<0.01 compared with the LPS group;

FIG. 14 shows gross specimens and HE stainings for mice kidney in each group, where A depicts the gross specimens of mice kidney, including a wild group, a model control group and a treatment group from left to right; B depicts HE stainings for mice kidney in each group (×200);

FIG. 15 shows kidney function and cyst-related indexes of mice, where A depicts kidney function of mice in each group; B depicts the cyst-related indexes of mice in each group, expressed as mean±SEM, ** means P<0.01 compared with the wild group; ## means P<0.01 compared to the model control group;

FIG. 16 shows immunohistochemical staining for Ki67 in mice kidney (×400);

FIG. 17 shows the levels of C3a in mice kidney tissue and serum, where A depicts C3a level in kidney tissues of mice in each group; B depicts C3a level in serum of mice in each group, expressed as mean±SEM, ** means P<0.01 compared with the wild group; # means P<0.05 compared with the model control group; ## means P<0.01 compared with the model control group;

FIG. 18 shows the relative expression of ADPKD-related signaling pathway protein in kidney tissue of mice in each group, wherein A depicts a Western blot image; B depicts a gray scale analysis image of protein expression in each group, expressed as mean±SEM, ** means P<0.01 compared with the wild group; ## means P<0.01 compared with the model control group.

### DETAILED DESCRIPTION OF THE INVENTION

The present disclosure provides a use of C3aR serving as drug target in preparing drug for preventing and/or treating polycystic kidney disease. The present disclosure has no special limitations on the types of polycystic kidney disease, comprising autosomal dominant polycystic kidney disease and autosomal recessive polycystic kidney disease, preferably autosomal dominant polycystic kidney disease. The drug of the present disclosure is preferably a drug capable of inhibiting the activity of C3aR and/or down-regulating the expression of C3aR gene.

The present disclosure also provides a use of an agent for inhibiting the activity of C3aR and/or down-regulating the expression of C3aR gene in preparing drugs for preventing or treating polycystic kidney disease. The present disclosure has no special limitations on the types of the agent for inhibiting the activity of C3aR and/or down-regulating expression of C3aR gene, and preferably includes, but is not limited to: proteins specifically binding to C3aR; SiRNA molecules, miRNA molecules or antisense nucleotides capable of specifically interfering expression and processing of C3aR gene; C3aR inhibitors, antagonists, downregulators, retardants or blockers. As a preferred example of the present disclosure, the C3aR inhibitor is preferably SB290157 having the chemical formula

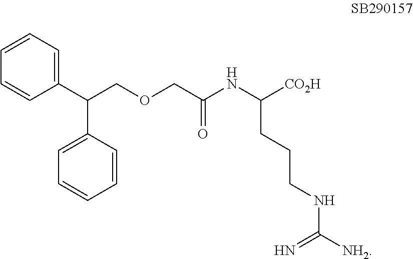 [SMILES: N=C(N)NCCCC(NC(=O)COCC(c1ccccc1)c1ccccc1)C(=O)O]

Preferably, the C3aR inhibitor of the present disclosure acts by any one or several of the following ways: inhibiting proliferation of cyst epithelial cells; blocking chemotaxis of C3a-C3aR on macrophages; inhibiting the interaction between C3a and C3aR; and lowering the C3aR level. More preferably, the C3aR inhibitor of the present disclosure down-regulates the expression of p-ERK and p-P65 proteins by inhibiting the interaction between C3a and C3aR and inhibiting the activation of ERK and NF-kB pathways.

The present disclosure also provides a use of C3aR as drug target in screening drugs for preventing and/or treating polycystic kidney disease, wherein the drug for preventing and/or treating polycystic kidney disease can inhibit the activity of C3aR and/or down-regulate the expression of C3aR gene.

The present disclosure also provides a drug for preventing and/or treating polycystic kidney disease, comprising an active ingredient capable of inhibiting the activity of C3aR and/or down-regulating the expression of C3aR gene. The active ingredient according to the present disclosure is preferably a C3aR inhibitor, more preferably SB290157. Further, the drug for preventing and/or treating polycystic kidney disease comprises an effective amount of a C3aR inhibitor and a pharmaceutically acceptable auxiliary material or carrier; the effective amount refers to an amount that is functional or active in humans and/or animals and that is acceptable to humans and/or animals. In a preferred embodiment of the present disclosure, the effective amount of C3aR inhibitor is administered at a dose of 0.1-10 mg/kg; the pharmaceutically acceptable adjuvant or carrier refers to an adjuvant or carrier for administration of a therapeutic agent, including various excipients and diluents, which are not necessary active ingredients in themselves and do not have excessive toxicity after administration. The present disclosure has no special limitations on the excipients and carriers, and both conventional pharmaceutical excipients and carriers in the field can be used. Further, the drugs of the present disclosure for preventing or treating polycystic kidney disease also include auxiliary substances, such as fillers, lubricants, glidants, wetting agents or emulsifiers, pH buffer substances, etc.

The technical solutions provided by the present disclosure are described in detail below with reference to the examples, but the technical solutions cannot be understood as limiting the protection scope of the present disclosure. In the following examples, various processes and methods that are not described in detail are conventional methods known in the art. The primers used are indicated for the first time and the same primers used thereafter are the same as those indicated for the first time. The methods used in the following examples are conventional methods unless otherwise specified.

### Example 1

Experimental animals: the animals used in this experiment were PKD1^{flox/flox}-Tamoxifen-Cre mice, gifted by professor Wüthrich RP, University of Zurich, Switzerland, and were bred in Specific Pathogen Free (SPF) Laboratory Animal Center of Naval Medical University [License No: SYXK (Shanghai): 2017-0004]. According to study of Klaus et al., PKD1^{flox/flox}-Tamoxifen-Cre mice were intraperitoneally injected with tamoxifen (dissolved in corn oil) at 15 mg/kg at postnatal day 10 to knock out Pkd1 gene, and littermate cre-negative mice served as negative controls.

Kidney tissues of patients: kidney tissues of ADPKD patients were obtained from the polycystic kidney surgically removed from Shanghai Changzheng Hospital. The tissues with cysts and cyst walls were collected and frozen in liquid nitrogen. The tissues of the unilateral kidney (from kidney cancer patients) removed more than 5 cm away from the side of cancer were taken as controls. All tissue specimens received informed consent from patients, and the study was approved by the Ethics Committee of Changzheng Hospital Affiliated to Naval Military Medical University (Approval number: 2017SL039).

The data was analyzed using SPS S24.0 statistical software. Normal distribution data was expressed as mean±standard deviation, while skewed distribution data was expressed as median and interquartile range. When the data obeyed normal distribution, t-test was used to compare the differences between the two groups, and variance analysis was used to compare the differences among multiple groups; when the data did not obey normal distribution, non-parametric test was used. Statistical significance was defined as P<0.05.

### Example 2

Comparison of C3a Levels in the Kidney Tissue and Serum of Wild-Type and Pkd1 Conditional Knockout Mice at Different Stages of Disease

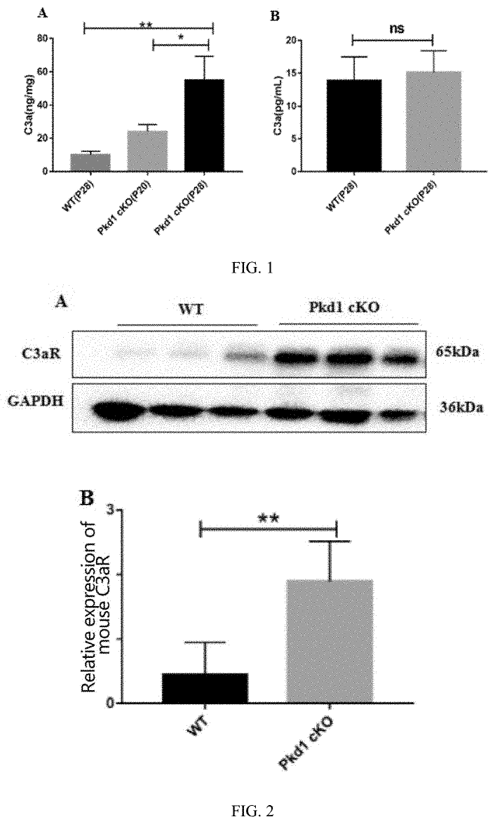

Serum and kidney tissues from Pkd1 conditional knockout mice at postnatal day 20 and 28 and wild-type mice at postnatal day 28 were taken out to prepare tissue homogenates, and C3a level was detected by mouse C3a Elisa kit (ElabscienceE-EL-M0337c, China) and human C3a Elisa kit (Elabscience, E-EL-H0818c, China). The results were shown in FIG. 1.

As can be seen from FIG. 1, compared with the wild-type mice at postnatal day 28 (10.11±1.20 ng/mg), the C3a level in the kidney tissue of simultaneous Pkd1 knockout mice (54.89±8.33 ng/mg) increased significantly, P<0.05; with the progression of polycystic kidney disease, the C3a level in the kidney tissue increased gradually, compared with Pkd1 knockout mice at postnatal day 20 (23.98±2.37 ng/mg), the C3a level in the kidney tissue of Pkd1 knockout mice at postnatal day 28 (54.89±8.33 ng/mg) increased significantly, P<0.05; however, there was no significant difference in serum C3a level between wild type mice (13.89±1.47 ng/ml) and Pkd1 knockout mice (15.09±1.49 ng/ml) at postnatal day 28 (P>0.05). It is suggested that the increase of C3a induced by complement activation in ADPKD may be mainly localized in the kidney rather than in the systemic system.

### Example 3

Comparison of the Expression of C3aR in the Kidney Tissue Between Wild-Type Mice and Pkd1 Conditional Knockout Mice

Western blot was used to detect the relative expression of C3aR protein in the kidney tissue of wild-type and Pkd1 conditional knockout mice. The results were shown in FIG. 2. As can be seen from FIG. 2, compared with wild type mice, the expression of C3aR in the kidney tissue of Pkd1 knockout mice was significantly up-regulated, P<0.05.

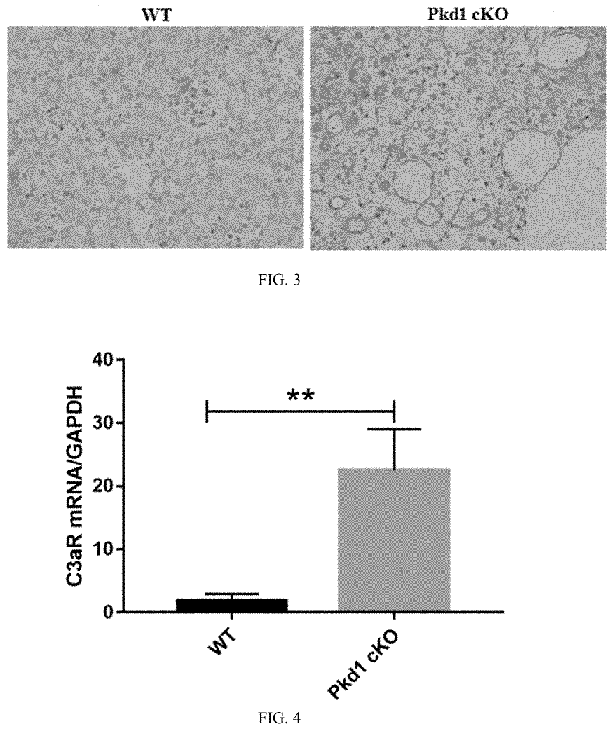

Immunohistochemistry was used to detect the expression of C3aR in the kidney tissue of wild-type and Pkd1 conditional knockout mice, and the results were shown in FIG. 3. As can be seen from FIG. 3, the number of cells positively stained for C3aR (brownish yellow) in the kidney tissue of Pkd1 knockout mice was significantly increased compared to wild type mice.

Real-time PCR was used to detect the relative expression of C3aR mRNA in the kidney tissue of wild-type and Pkd1 conditional knockout mice. In the real-time PCR detection, a 20 μl reaction system is provided, mainly including: 10 μl SYRB-PCR-MIX, 7.2 μl deionized water, 0.4 μl primer F, 0.4 μl primer R, and 2 μl cDNA template; the RT-PCR reaction procedure was as follows: stage 1: 95° C. for 1 min, stage 2: 95° C. for 15 s, 60° C. for 31 s for 45 cycles, stage 3: 95° C. for 15 s, 60° C. for 15 s, 95° C. for 15 s. C3aR primer sequence Mouse F: 5′-CAGGGAAAAGTCAGTGCTCAG-3′ (SEQ ID NO:1), Mouse R: 5′-GGCCGTGAGTGTAGGTCAGT-3′ (SEQ ID NO:2), GAPDH primer sequence Mouse F: 5′-AGAACATCATCCCTGCATCC-3′ (SEQ ID NO:3), Mouse R: 5′-ATACCAGGAAATGAGCTTGAC-3′ (SEQ ID NO:4). The data was analyzed and the CT value referred to the number of cycles required for the gene to be tested to achieve exponential amplification. The CT values in the 3 auxiliary wells were averaged, GAPDH was set as an internal reference, and a relative quantitative method was used to calculate the expression fold of the target gene relative to GAPDH=2^{-ΔΔ}^{^{CT}}. ΔΔCT referred to the difference value between the mean value of the target gene CT and the mean value of GAPDH CT. The results were shown in FIG. 4. As can be seen from FIG. 4, the expression of C3aR mRNA was significantly up-regulated in the kidney tissue of Pkd1 knockout mice compared to wild type mice, P<0.05.

### Example 4

Comparison of the Expression of C3aR in the Kidney Tissue Between Normal Human and ADPKD Patients

Western blot was used to detect the relative expression of C3aR protein in the kidney tissues of normal human and ADPKD patients, and gray scale analysis was performed. The results were shown in FIG. 5. As can be seen from FIG. 5, the expression of C3aR protein was significantly up-regulated in the kidney tissue of ADPKD patients compared to normal human, P<0.05.

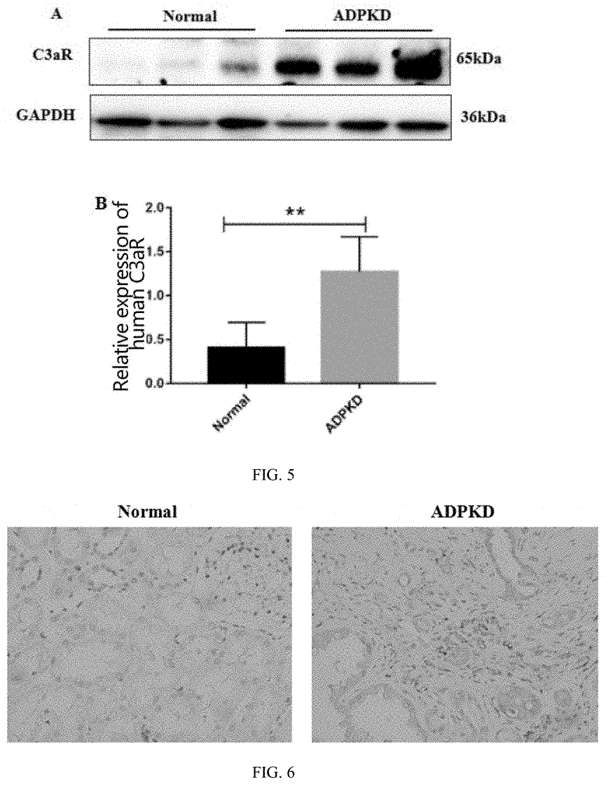

Immunohistochemistry was used to detect the expression of C3aR in the kidney tissue of normal human and ADPKD patients, and the results were shown in FIG. 6. As can be seen from FIG. 6, the number of cells positively stained for C3aR (brownish yellow) was significantly increased in ADPKD patients compared to normal human.

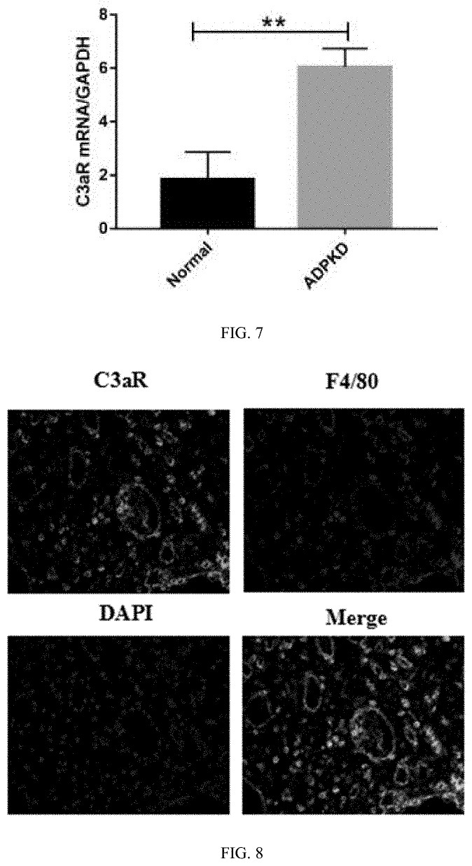

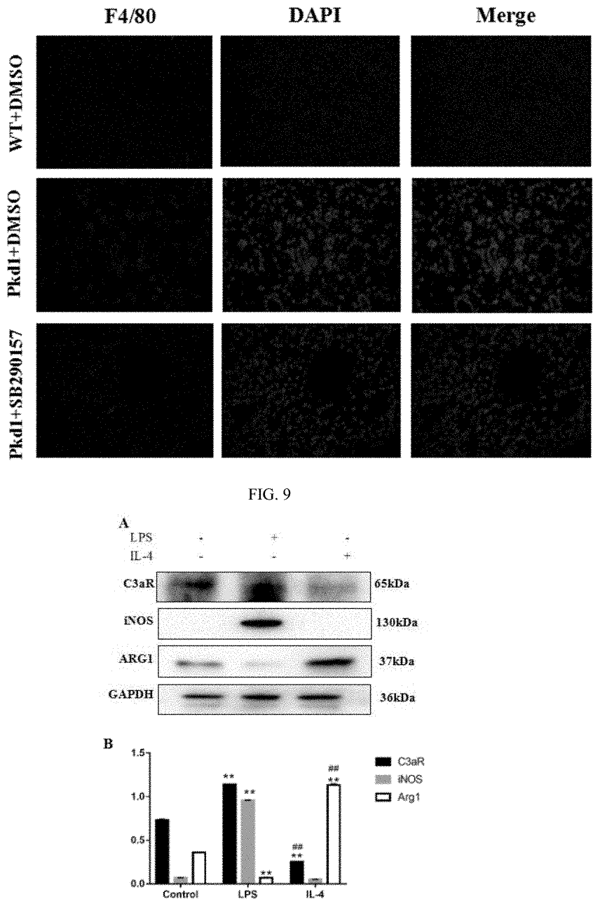

RT-PCR was used to detect the relative expression of C3aR mRNA in the kidney tissues of normal human and ADPKD patients. In the real-time PCR detection, the reaction system, the reaction procedure and the data analysis were the same as those in Example 3, C3aR primer sequence Human F: 5′-CCCACTGTCCCTCAAACAAT-3′ (SEQ ID NO:5), Human R: 5′-AAGTCCGCTGCTCACCATA-3′ (SEQ ID NO:6), GAPDH Primer Sequence Human F: 5′-GGAAACTGTGGCGTGATG-3′ (SEQ ID NO:7), Human R: 5′-TGGGTGTCGCTGTTGAAG-3′ (SEQ ID NO:8). The results were shown in FIG. 7. As can be seen from FIG. 7, the relative expression of C3aR mRNA was significantly up-regulated in the kidney tissue of ADPKD patients compared to normal human, P<0.05.

### Example 5

Observation of the Localization of C3aR and Macrophage Marker F4/80 in the Kidney Tissue of Pkd1 Knockout Mice

F4/80 was a cell surface glycoprotein that is widely expressed in various mature macrophages in mice, such as the macrophages in Kupffer cells, Langerhans cells, microglia and macrophages in bone marrow stroma, thymus, etc. In order to further clarify the expression site of C3aR, C3aR and F4/80 in the kidney tissues of Pkd1 knockout mice were co-stained by immunofluorescence, and the expression of C3aR in the infiltrated macrophages (RAW 264.7 cells: Murine bone marrow-derived macrophages, purchased from Wuhan Boster Biological Technology., Ltd.) in polycystic kidney tissues were observed. The results were as shown in FIG. 8, it can be seen from FIG. 8 that there was co-localization of C3aR with F4/80 (yellow), indicating that C3aR was expressed on the macrophage membrane.

### Example 6

Observation of the Effect of C3aR Inhibitor on Macrophage Infiltration in the Kidney Tissues of Pkd1 Conditional Knockout Mice

The mice were divided into three groups, including WT+DMSO (a wild group), Pkd1+DMSO (a model control group), and Pkd1+SB290157 (1 mg/kg, intraperitoneal injection, once a day, a treatment group). F4/80 in the kidney tissues of the wild group, the model control group and the SB290157 treatment group were stained by immunofluorescence to compare the macrophage infiltration. The results were as shown in FIG. 9, it can be seen from FIG. 9 that the positive rate of F4/80 in the kidney tissue of the model control group was higher than that in the wild group, while the positive rate in the treatment group was significantly lower than that in the control group, indicating that the inhibition of C3aR could reduce macrophage infiltration in the kidney tissue of Pkd1 knockout mice and reduce the inflammatory state, which suggested that C3a-C3aR may have chemotactic effects on ADPKD kidney macrophages.

### Example 7

Observation of the Expression of C3aR in Macrophages of Different Polarization Types

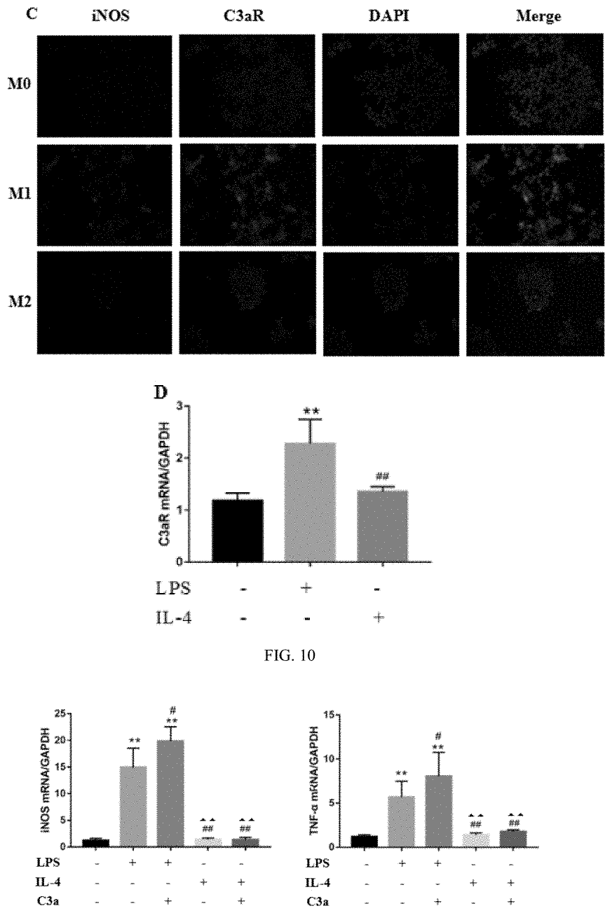

Macrophages in different microenvironments may be polarized to different phenotypes: M1-type and M2-type, with unpolarized macrophages as M0-type. In order to further clarify the expression of C3aR in macrophages of different polarization types, RAW 264.7 cells were divided into three groups including a control group (M0-type), a 100 ng/ml LPS-stimulation group (inducing macrophage being polarized to M1-type), and a 20 ng/ml IL-4 stimulation group (inducing macrophage being polarized to M2-type). After 24 hours, cell RNA and protein were extracted, Western blot was used for detecting the expression of C3aR, iNOS (M1-type macrophage marker) and ARG1 (M2-type macrophage marker) in each group of cells, RT-PCR was used for detecting the expression of C3aR mRNA in each group of cells, and immunofluorescence was used for comparing the expression of C3aR in the three types of macrophages. The results were shown in FIG. 10, it can be seen from FIG. 10 that the expression of C3aR in M1-type macrophages was significantly up-regulated at both protein and RNA levels compared to M0-type and M2-type macrophages, P<0.05.

### Example 8

Observation of the Effect of C3a Stimulation on the Expression of Inflammatory Factors Such as Macrophage Markers and TNF-α

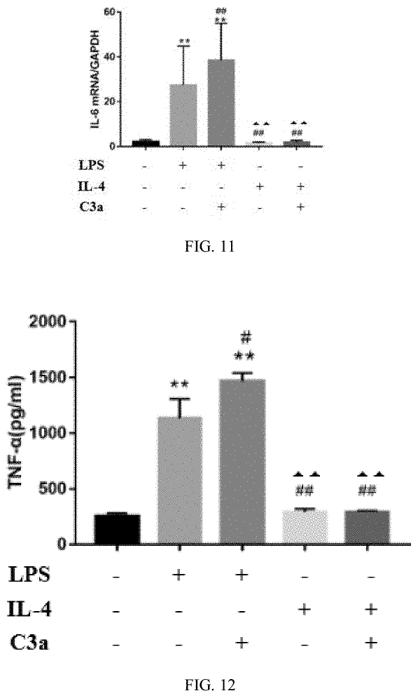

The C3aR expression on the cell surface affected the sensitivity of downstream signals induced by C3a, so C3a-C3aR should act mainly on M1-type macrophages, which was consistent with the downstream pro-inflammatory effect. To verify the effect of C3a stimulation on the expression of M1-type macrophage inflammatory factor, RAW264.7 cells were divided into 5 groups: (1) a control group: without any stimulation; (2) a LPS group: stimulated with 100 ng/ml LPS; (3) a LPS+C3a group: stimulated with 100 ng/ml LPS+100 ng/ml C3a; (4) a IL-4 group: stimulated with 20 ng/ml IL-4; (5) a IL-4+C3a group: stimulated with 20 ng/ml IL-4+100 ng/ml C3a. After 24 hours, cell RNA was extracted, and RT-PCR was used for detecting iNOS, TNF-α and IL-6 mRNA level in each group of cells, wherein the primer sequence used by RT-PCR was as follows: iNOS primer sequence Mouse F: 5′-TGGAGCGAGTTGTGGATTGTC-3′ (SEQ ID NO:9), Mouse R: 5′-GTGAGGGCTTGGCTGAGTGA-3′ (SEQ ID NO:10), IL-6 primer sequence Mouse F: 5′-TCCGGAGAGGAGACTTCACA-3′ (SEQ ID NO:11), Mouse R: 5′-TCCAGTTTGGTAGCATCCATCA-3′ (SEQ ID NO:12), TNF-α primer sequence Mouse F: 5′-GGCATGGATCTCAAAGACA-3′ (SEQ ID NO:13), Mouse R: 5′-TGGGCTCATACCAGGGTT-3′ (SEQ ID NO:14). The results were shown in FIG. 11, iNOS mRNA level of M1-type macrophage surface marker could be up-regulated by C3a stimulation, P<0.05, and the mRNA level of main inflammatory factors including TNF-alpha and IL-6 produced by M1-type macrophages were remarkably up-regulated, P<0.05. In contrast, C3a had no significant pro-inflammatory effect on M2-type macrophages, P>0.05.

The TNF-α level in the culture supernatants of macrophages from different treatment groups were detected by Elisa. The results were shown in FIG. 12, the TNF-α level secreted by M1-type macrophages was significantly higher than that of M0-type and M2-type macrophages, and the TNF-α level secreted by M1-type macrophages was further increased after C3a stimulation (P<0.05). Therefore, C3a stimulation not only promoted the expression of autoinflammatory factors in M1-type macrophages, but also affected the surrounding inflammatory microenvironment.

### Example 9

Observation of the Effect of C3a Stimulation on Macrophage-Associated Signaling Pathway

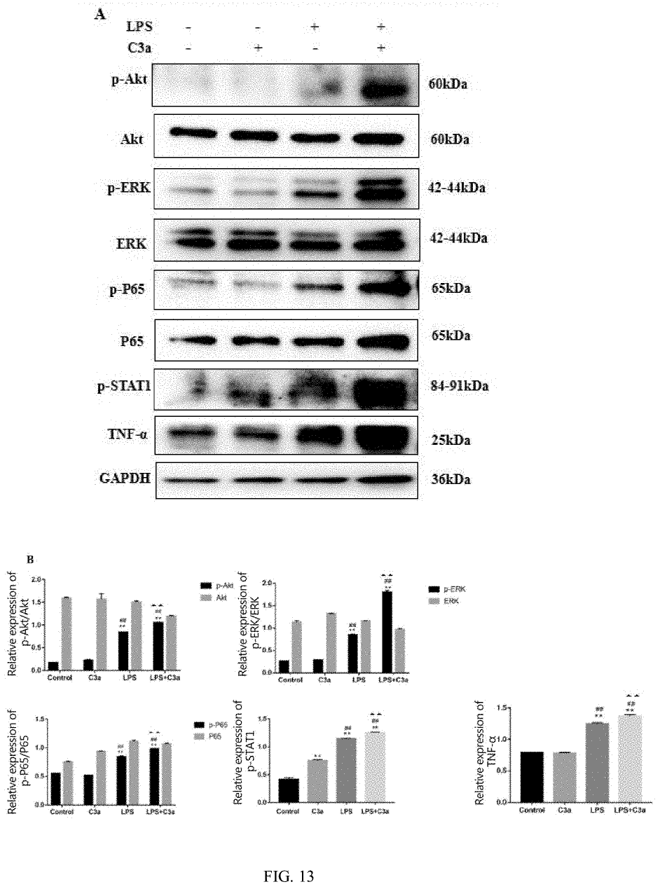

In order to explore the mechanism of pro-inflammatory effects of C3a-C3aR on M1-type macrophages, RAW264.7 cells were divided into 4 groups with LPS as a stimulation factor and C3a as an intervention factor: (1) a control group (without any stimulation); (2) a C3a (100 ng/ml) stimulation group; (3) a LPS (100 ng/ml) stimulation group; (4) a C3a (100 ng/ml)+LPS (100 ng/ml) stimulation group. After 24 hours, cell proteins in each group were extracted, and Western blot was used for detecting the expression of p-Akt, p-ERK, p-P65, p-STAT1 and TNF-α. The results were shown in FIG. 13, compared to M0-type macrophages, the levels of p-Akt, p-ERK, p-P65, p-STAT1 and TNF-α proteins in cells were significantly up-regulated after orientation polarization of macrophages to M1-type by LPS stimulation, P<0.05, and related signal pathways were over-activated; after M1-type macrophages stimulated by C3a, the expression of these proteins was further up-regulated, P<0.05; in contrast, the stimulatory effect of C3a on M0-type macrophages was not significant, P>0.05. Akt and ERK may regulate the apoptosis and proliferation of M1-type macrophages, while NF-κB and STAT1 was an important signaling pathway for polarization and related inflammation of M1-type macrophage. Therefore, it is speculated that C3a-C3aR may promote the activation of Akt, ERK, STAT1 and NF-κB pathway in M1-type macrophages, and then promote the secretion of TNF-α inflammatory factors by M1-type macrophages.

### Example 10

Observation of the Effect of C3aR Inhibitor SB290157 on the Cyst Indexes and Kidney Function of Pkd1 Knockout Mice by In Vivo Experiments

The mice were divided into three groups, including WT+DMSO (a wild group), Pkd1+DMSO (a model control group), Pkd1+SB290157 (1 mg/kg, intraperitoneal injection, once a day, a treatment group). The mice were intraperitoneally injected with tamoxifen dissolved in corn oil to knock out Pkd1 gene at postnatal day 10, then administrated at postnatal day 12, and sacrificed at postnatal day 28. Blood and kidney specimens were collected from mice. The blood creatinine, urea nitrogen, kidney/body weight, and cyst index of each group of mice were compared.

The main reagents used were a C3aR inhibitor (Calbiochem, SB290157, Germany), Creatinine Assay Kit (Nanjing Jiancheng Bioengineering Institute), Urea Nitrogen Assay Kit (Nanjing Institute of Built Bioengineering), and dimethyl sulfoxide (DMSO) (Amresco, 1963C070, USA).

Serum creatinine (μmol/L)=[(Sample A2-K*Sample A1)−(Blank A2-K*Blank A1)]/[(Standard A2-K*Standard A1)−(Blank A2-K*Blank A1)]*Standard Concentration; Formula:

Urea nitrogen (mmol/L)=(Assay OD−Blank OD)/(Standard OD−blank OD)*Standard Concentration (10 mmol/L)*Dilution multiple;

After HE staining, scanning was performed with a whole slide image scanner, and pictures were exported and saved using CaseViewer software. The total pixel value of the whole kidney in Photoshop software was calculated as A1, a cyst area was selected and calculated the total pixel value as A2, wherein the ratio of A2 to A1 is the cyst index.

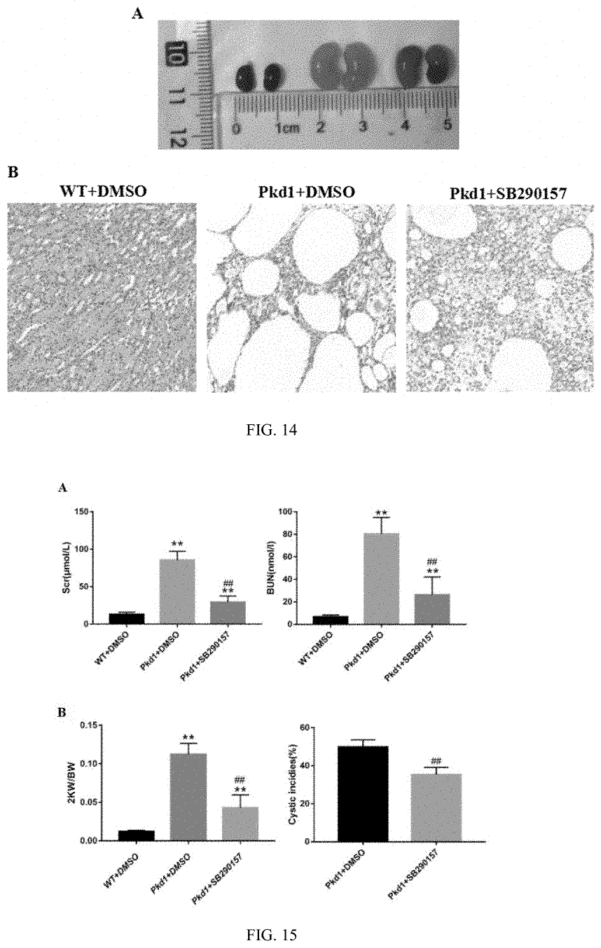

The results were shown in FIGS. 14 and 15, the kidney function and cyst-related indexes of the mice in each group were compared. Compared with the model control group, the kidney volume of the mice in the treatment group was reduced, and HE staining showed a reduction of the cyst area. After whole slide scanning, the cyst index was calculated, wherein, the treatment group (35.3±1.9%) was significantly lower than the control group (49.9±1.9%), P<0.05; the double kidney weight/body weight ratios (2KW/BW) in the control group (0.112±0.006) and the treatment group (0.043±0.007) were significantly higher than the wild group (0.012±0.000), P<0.05, and the treatment group was significantly lower than the control group (P<0.05); the serum creatinine in the control group (90.0±3.6 μm) and the treatment group (29.3±3.4 μm) were significantly higher than the wild group (13.0±1.2 μmol/L), P<0.05, and the treatment group was significantly lower than the control group (P<0.05); the blood urea nitrogen in the control group (80.4±6.0 mmol/L) and the treatment group (26.3±6.5 mmol/L) were significantly higher than the wild group (6.8±0.7 mmol/L), P<0.05, the treatment group was significantly lower than the control group (P<0.05).

### Example 11

Inhibition of Proliferation of Cystic Cells by SB290157

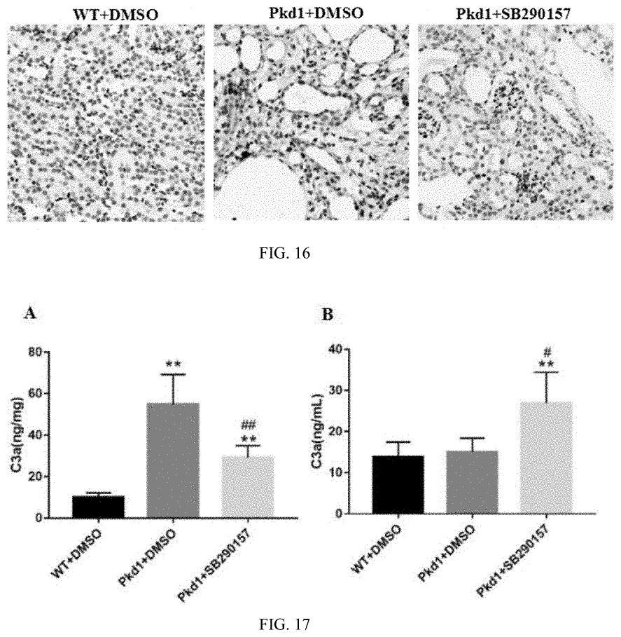

In order to explore whether the proliferation of mouse cyst epithelial cells was restricted after treatment, immunohistochemical staining for Ki67 was performed on kidney slices of each group of mice, wherein Ki67 was a nuclear protein that could reflect the cell proliferation level. The results were shown in FIG. 16, the number of kidney Ki67 positive staining (brown) cells in the model control group and the treatment group was higher than that in the wild group, and the treatment group was significantly lower than that in the model control group. The results showed that SB290157 could inhibit the proliferation of mouse cyst epithelial cells and inhibit the growth of cyst.

### Example 12

Reduction of the C3a-C3aR Level and Inhibition of the Activity of Several ADPKD-Related Signaling Pathways in Pkd1 Conditional Knockout Mice Kidney by SB290157

The mice were divided into three groups, including WT+DMSO (a wild group), Pkd1+DMSO (a model control group), Pkd1+SB290157 (1 mg/kg, intraperitoneal injection, once a day, a treatment group). The mice were intraperitoneally injected with tamoxifen dissolved in corn oil to knock out Pkd1 gene at postnatal day 10, then administrated at postnatal day 12, and sacrificed at postnatal day 28. Blood and kidney specimens were collected from mice. Elisa was used to detect the levels of kidney tissue homogenate and serum C3a in each group. As shown in FIG. 17, compared with the model control group (54.89±8.33 ng/mg), the C3a level in the kidney tissue in the treatment group (29.17±2.21 ng/mg) was significantly decreased, P<0.05, but it was still higher than that in the wild group (10.11±1.20 ng/mg), P<0.05. This was because SB290157 inhibited the interaction between C3a and C3aR in the local mouse kidney and reduced the production of C3a. Compared with the control group (15.09±1.49 ng/ml), the C3a level in serum in the treatment group (26.91±3.38 ng/ml) was increased, P<0.05. This was because SB290157 extensively inhibited C3aR in the systemic system, resulting in accumulation and compensatory increase of C3a in the circulation.

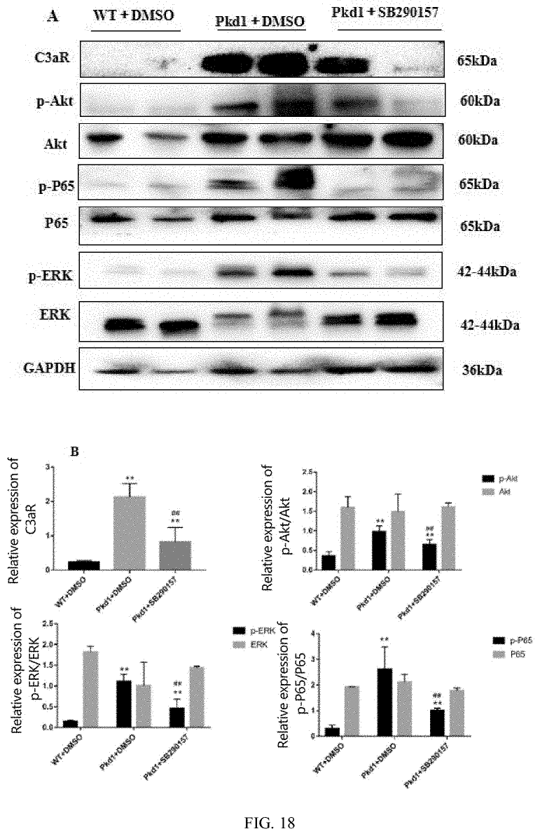

In order to further explore the therapeutic mechanism of SB290157 on polycystic kidney mice, the expression of ADPKD-related pathway protein was detected by Western blot. The results were shown in FIG. 18, the expression of p-Akt, p-ERK, and p-P65 protein was down-regulated in the treatment group compared to the model control group, suggesting that the proliferation of polycystic kidney tissue was restricted and the inflammatory state was improved in the treatment group. In addition, SB290157 also reduced the C3aR level.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for preventing and/or treating polycystic kidney disease, wherein the C3aR is used as drug target in the therapeutic drug.

2. The method according to claim 1, wherein the polycystic kidney disease comprises autosomal dominant polycystic kidney disease and autosomal recessive polycystic kidney disease.

3. The method according to claim 1, wherein the therapeutic drug is capable of inhibiting the activity of C3aR and/or down-regulating the expression of C3aR gene.

4. A method for preventing or treating polycystic kidney disease, wherein comprising using an agent in the method for preventing or treating polycystic kidney disease.

5. The method according to claim 4, wherein the agent includes but is not limited to:
proteins specifically binding to C3aR;
siRNA molecules, miRNA molecules or antisense nucleotides capable of specifically interfering expression and processing of C3aR gene;
C3aR inhibitors, antagonists, downregulators, retardants or blockers.

6. The method according to claim 5, wherein the C3aR inhibitors comprise SB290157 and compounds with similar or identical activity and function to SB290157.

7. The method according to claim 5, wherein the C3aR inhibitor acts through any one or several of the following ways:
inhibiting proliferation of cyst epithelial cells; blocking chemotaxis of C3a-C3aR on macrophages; inhibiting the interaction between C3a and C3aR; and lowering the C3aR level.

8. The method according to claim 7, wherein the C3aR inhibitor down-regulates the expression of p-ERK and p-P65 proteins by inhibiting the interaction between C3a and C3aR and inhibiting the activation of ERK and NF-κB pathways.

9. A method for preventing and/or treating polycystic kidney disease, wherein the C3aR is used as drug target for screening the therapeutic drug, and the drugs for preventing and/or treating polycystic kidney disease is capable of inhibiting the activity of C3aR and/or down-regulating the expression of C3aR gene.

10. A drug for preventing and/or treating polycystic kidney disease, comprising an active ingredient capable of inhibiting the activity of C3aR and/or down-regulating the expression of C3aR gene.
